(19) United States
(12) Patent Application Publication
(10) Pub. No.: US 2014/0220103 A1
(43) Pub. Date: Aug. 7, 2014

(54) HEMOSTATIC DRESSING FOR ARTERIAL BLEEDING

(71) Applicant: The Trustees of Columbia University in the City of New York, New York, NY (US)

(72) Inventors: Matthew D. Bacchetta, New York, NY (US); Gopal Singh, Oakland Gardens, NY (US); Maurizio A. Miglietta, New York, NY (US)

(73) Assignee: The Trustees of Columbia University in the City of New York, New York, NY (US)

(21) Appl. No.: 14/250,828

(22) Filed: Apr. 11, 2014

Related U.S. Application Data
(63) Continuation of application No. PCT/US12/60054, filed on Oct. 12, 2012.
(60) Provisional application No. 61/546,527, filed on Oct. 12, 2011.

(51) Int. Cl. A61L 15/28; A61L 15/32; A61L 15/38; A61L 15/18

(52) U.S. Cl. CPC A61L 15/28; A61L 15/18; A61L 15/32; A61L 15/38
USPC 424/445; 424/94.64; 424/447

(57) ABSTRACT

A wound treatment system having a formulation to retard bleeding. In particular, the wound treatment system includes medical device, e.g. bandage, which can be adapted for internal or external use. The wound treatment system includes a fabric substrate having a formulation, wherein the formulation includes chitosan, kaolin, fibrinogen, and thrombin.

### BACKGROUND OF THE DISCLOSED SUBJECT MATTER

1. Field of the Disclosed Subject Matter

The disclosed subject matter relates to a novel hemostatic dressing incorporating clotting factors and absorbent compounds for hemostasis and wound treatment.

2. Description of Related Art

Hemorrhagic events, from the minor to the life threatening, result from a wide variety of circumstances and occur in a wide variety of settings. The conditions which result in hemorrhage may be relatively predictable, such as those associated with medical procedures. Alternatively, hemorrhagic events may result from unpredictable circumstances, such as a breach of the skin or an internal organ in an accident. Such acute traumatic wounds occur in an almost infinite number of patterns and degrees, making the use of simple compression or application of a single type of bandage, impractical if not impossible, especially in the most severe circumstances. For example, a traumatic wound to the groin cannot be readily controlled either by simple direct pressure or by the use of a simple flat bandage.

A variety of methods and products are known which partially address the treatment of hemostasis, and/or the need for flexibility in wound dressings. For example, Hemcon's Chitosan Bandage provides a gauze bandage impregnated with chitosan. Chitosan, a fiber derived from chitin in shellfish, is a nondigestible aminopolysaccharide. Chitosan is generally synthesized by removing acetyl groups from chitin, through a process called deacetylation. Chitosan is known to have significant coagulant properties which are attributable to its cationic (positive charge) and/or mucoadhesive properties. Numerous studies have demonstrated that in models of life threatening hemorrhage, the ability of the bandage to improve survival has been limited (see, J Trauma 2005; 59:865-875 and J Trauma 2004; 56:974-983). In one study, use of the bandage had a 100% failure rate when employed in an isolated arterial injury. In another study (which combined arterial and venous hemorrhage at low blood pressures) the bandage resulted in a 28% mortality rate. It was noted that there was a bandage-to-bandage variability in performance and ability of the bandage to adhere to the wound. Accordingly, there remains the need for the ability to produce a powder or granular form of chitosan for wound treatment which addresses the tendency of powdered chitosan to resist mixing with blood.

Another commercially available wound treatment product is the Fibrin Sealant Dressing (FSD) which is the result of a collaborative effort between the U.S. Army and the American Red Cross. The FSD is made from fibrin, thrombin, and factor XIII purified from human donated blood and plasma. The FSD is thus a biologic which has a potential for disease transmission. The FSD controls hemorrhage by promoting natural clot formation at the site of injury since it provides concentrated coagulation factors at the site of injury. However, it is a biologic and the manufacture of such bandages is extremely labor-intensive, and thus cost prohibitive in most circumstances. Another drawback is that these dressings are only available in bandage form which are fragile and tend to break apart if not carefully handled.

Another commercially available wound treatment product is the Rapid Deployable Hemostat (RDH) which is a bandage made by Marine Polymer Technologies and incorporates a derivative from sea algae to promote hemostasis. However, in a study by Alam and colleagues (Alam, et al. J Trauma 2003; 54:1077-1082), which explored the ability of many commercial products to stop severe bleeding and to increase survival, use of the RDH resulted in lower survival rates than a simple standard bandage. Consequently, and as this study indicates, the current components of the RDH are not suitable for use in life threatening hemorrhage. Again, the RDH is only available in bandage form and the costs associated with these bandages are high which can limit their applicability in most circumstances.

Yet another known wound treatment system is provided in U.S. Pat. No. 4,748,978 (to Kamp) which discloses a therapeutic dressing that includes a flexible permeable support and a mixture of mineral components, including bentonite, kaolinite and illite or attapulgite, and may include anti-fungal (or other) agents as well. The dressing is reported to be designed to be flexible and to be able to be made or cut to any desired size. Additionally, the dressing reports to be intended primarily to treat burns, but can also be used for the treatment of ulcers. However, the dressing is not described as suitable for the treatment of hemorrhage, and no data from Kamp is available to support its use for this indication.

Still another known wound treatment system is provided in U.S. Pat. No. 4,822,349 (to Hursey et al.) which describes a non-bandage material used to treat bleeding. The material is sold by Z-Medica as “Quick-Clot” (available at www.z-medica.com) and is a granular form of zeolite, an aluminum silicate mineral. During use, it is poured into a wound. In addition to absorbing water from hemorrhaged blood and concentrating hemostatic factors in the blood at the site of injury, its mechanism of action appears to involve chemical cautery. An intense exothermic reaction is produced upon contact with liquid (e.g. blood), and is likely responsible for stoppage of blood flow by cauterization. While use of this material may be preferable to bleeding to death, the attendant burning of tissue at and near the wound (and possible burn injury of medial personnel who are administering the material) is clearly a severe disadvantage. This side effect also reduces the ability of the material to be used for internal hemorrhage. While the manufacturer indicates that the main mechanism of action is the superabsorbent nature of zeolite which absorbs water out of blood to concentrate clotting factors, the '349 patent indicates that its action lies mainly through the exothermic reaction it creates. Studies by Alam and colleagues (J Trauma 2004; 56:974-983) clearly demonstrate that the ability of this product to stop hemorrhage is quickly lost when it is partially hydrated in attempts to reduce the exothermic reaction and the resulting temperature it produces in tissues. Indeed, when the granules are placed in a bag similar to a tea bag to facilitate removal, its ability to stop bleeding is significantly limited. Additionally, this product is not commercially available as a bandage, and even if it were, it would likely still produce a significant exothermic reaction upon contact with blood.

Another commercially available wound treatment made by TraumaDex which is embodied as a non-bandage. In this case, the product is a powder consisting of microporous beads which absorb water and which contain concentrated clotting factors. During use, the material is poured or squirted into the wound. However, when studied by Alam and colleagues (J Trauma 2003; 54:1077-1082) in a model of severe hemorrhagic shock, TraumaDex performed no better than a standard field dressing, thus offering no advantage and certainly more expense. Alam and colleagues studied this product again (J Trauma 2004; 56:974-983) and demonstrated its performance to be suboptimal compared to QuickClot and the Hemcon bandage. In this study, it performed only slightly better than a standard dressing. Additionally, this product is not commercially available as a bandage, and even if it were, it would likely lack efficacy in stopping severe bleeding.

Thus, there remains a need for an efficient and effective wound treatment method and product that incorporates potentiators of clotting and absorbent compounds to create a dressing that effectively controls severe bleeding by providing local hemostasis.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

The disclosed subject matter includes a medical device including a fabric substrate including a formulation, wherein the formulation includes chitosan, kaolin, fibrinogen, and thrombin. In one embodiment the fibrinogen is in an amount of about 25 to 50 grams, the thrombin is in an amount of about 1000 to 2000 kilo units, the kaolin is in an amount of about 2 grams to 10 grams, and the chitosan is in an amount of about 4 grams to 10 grams. In another embodiment the fibrinogen is a fibrinogen buffer solution, and the fibrinogen buffer solution further includes tris(hydroxymethyl)aminomethane (TRIS), sodium chloride, sucrose, human serum (e.g. albumin), and Tween. In another embodiment the thrombin is a thrombin buffer solution, and the thrombin buffer solution includes sodium chloride, TRIS, calcium chloride, Lysine, and human serum. In yet another embodiment, the kaolin is a kaolin solution, and the kaolin solution includes phosphate buffered saline (PBS). In yet another embodiment, the chitosan is a chitosan solution, and the chitosan solution further includes PBS. In yet another embodiment, the formulation has a pH of about 6 to 7, e.g., 7.4. In some embodiments, the substrate is a dressing, while in others the substrate is configured as a bandage. In certain embodiments the fabric substrate comprises a single sheet of a fabric. In another embodiment, the fabric substrate comprises a composite textile. In another embodiment the fabric substrate has a size of approximately 1 to 3 inches in length and approximately 3 to 5 inches in width. In still another embodiment, the fabric substrate has a size of approximately 6 to 9 inches in length and approximately 8 to 11 inches in width.

The disclosed subject matter also includes a method of reducing bleeding from a wound site including providing a medical device comprising a fabric substrate and a formulation comprising kaolin, fibrinogen, thrombin, and chitosan; and applying the medical device to the wound site. In another embodiment, there is disclosed a method for fabricating a hemostatic dressing, including providing a fabric substrate, applying a kaolin solution to the fabric substrate, applying a chitosan solution to the fabric substrate, applying a fibrinogen solution to the fabric substrate, and applying a thrombinogen solution to the fabric substrate. In one embodiment, the method of producing the fibrinogen solution includes dissolving in 100 milliliter of 1% PBS: 16.76 grams of 100 millimolar TRIS Buffer, 2.94 grams of 10 millimolar sodium chloride 15 grams of 1.5% sucrose, 16 grams of 80 milligrams per gram human serum, and 3.0 g 15 milligrams per gram Tween. In another embodiment, the method of producing the kaolin solution includes dissolving 10 grams of kaolin in 200 ml of PBS. In yet another embodiment, the method of producing the chitosan solution includes dissolving 10 grams of chitosan in 200 milliliters of PBS, adding hydrochloric acid to achieve a pH of less than 6.5, heating at 70 degrees centigrade for forty minutes, adding sodium hydroxide to achieve a pH of 7.4, and allowing the chitosan solution to cool. In yet another embodiment, the method includes placing the substrate in a receptacle, applying 2 grams of the kaolin solution to the substrate, applying 4 grams of the chitosan solution to the substrate, maintaining the substrate at minus eighty degrees centigrade for at least a first fifteen minutes, applying 10 grams of the fibrinogen solution to the substrate, maintaining the substrate at minus eighty degrees centigrade for at least a second fifteen minutes, applying 100 kilo units of the thrombin solution to the substrate, and maintaining the sheet at minus eighty degrees centigrade for at forty-five minutes.

The disclosed subject matter also includes a formulation useful to stop bleeding from a wound including chitosan, kaolin, fibrinogen, and thrombin. In one embodiment, the fibrinogen is a fibrinogen buffer solution, and the fibrinogen buffer solution further includes TRIS, sodium chloride, sucrose, human serum and Tween. In another embodiment, the thrombin is a thrombin buffer solution, and the thrombin buffer solution further includes sodium chloride, TRIS, calcium chloride, lysine and human serum. In another embodiment, the thrombin is a thrombin solution, and the thrombin solution further includes glycine and glucose. In yet another embodiment, the kaolin is a kaolin solution further including PBS. In yet another embodiment, the chitosan is a chitosan solution further including PBS.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

FIG. 1 is a representation of the thrombin/fibrin clotting cascade.

FIGS. 2A-2C are graphs of test results from using the subject matter of the present disclosure.

### DETAILED DESCRIPTION OF THE EXEMPLARY EMBODIMENT

Reference will now be made to the exemplary embodiments of the disclosed subject matter. The device and corresponding steps of the disclosed subject matter will be described in conjunction with the detailed description of the system.

Uncontrolled hemorrhage is the most common cause of death on the battlefield and the second leading cause of death in civilian trauma patients and the search for efficacious treatment in pre-hospital settings remains one of the most challenging problems facing emergency medical professionals. In the pre-hospital phase, hemostatic agents can potentially reduce morbidity and mortality through the early control of hemorrhage. Early control of hemorrhage is imperative. Even with adequate resuscitation, significant blood loss leads to hypothermia, coagulopathy, acidosis, and late “second-hit” mortality that occurs through the development of sepsis and multiple organ failure. When hospital resuscitation includes blood transfusion, mortality increases independent of shock severity. Therefore, it is important to create a hemostatic agent capable of gaining control of early hemorrhage.

The blood coagulation cascade is an exquisite example of a responsive self assembly process in biology. When a wound is formed, a cascade of self-assembly events occurs in blood at the site of the wound. The net outcome is the assembly of the globular protein, fibrinogen, catalyzed by a second protein, thrombin to yield chains of fibrin. A network of insoluble fibrin chains forms the hemostatic “plug” or clot, which presents a physical barrier to the loss of blood from the wound. The coagulation cascade is a delicately balanced series of events—if it was to occur too easily, blood clots may form in unwanted areas leading to strokes or other complications. Scientists have long sought to harness the clotting power of fibrin to create hemostatic dressings or bandages. Hemostatic dressings that can staunch the bleeding from serious wounds are a pressing need both in civilian trauma centers as well as for military personnel. Indeed, uncontrolled hemorrhage from severe injuries is a leading cause of death among young adults (e.g., accident victims) and it is also responsible for the majority of deaths on the battlefield.

In accordance with the disclosed subject matter, a unitary or plurality of dressings are placed into an appropriate sized mould(s). Thereafter, 4 milliliters of 2% sucrose is pipetted on top of the boundary (e.g. edges) of the dressing(s). Once completed, the dressing will be placed in an 80 degree centigrade freezer for 60-75 min.

A formulation of Fibrinogen is made in a 100 mM tris(hydroxymethyl)aminomethane (TRIS) buffer, 10 mM Sodium Chloride, 1.5% Sucrose, FBS or Human Serum (e.g. Albumin) at a concentration of 80 mg/g and 15 mg/g of Tween. The fibrinogen concentration is adjusted to 50-mg/g using (Complete fibrinogen buffer CFB). The final pH of the fibrinogen is 7.4±0.1. Once prepared the fibrinogen is placed on ice until use.

Thrombin is formulated in 150 mM Sodium Chloride, 10 mM TRIS, 40 mM Calcium Chloride, 100 mM L-Lysine with the addition of HAS at 10 Oug/ml (Complete thrombin Buffer CTB), and 100 mg/g FBS. The final pH of the Thrombin is 7.4±0.1. Once prepared the thrombin is placed on ice until use.

A Kaolin solution is prepared using kaolin powder dissolved in a 1% phosphate buffered saline or an appropriate solubalizing agent for approximately 15 minutes prior to the application of fibrinogen and thrombin. In some embodiments, it can be advantageous to prevent this solution from standing for an extended period of time as this solution is prone to settle or agglomerate. In some embodiments, a range of approximately 2-5 grams of kaolin is used for each dressing.

A chitosan solution is made using chitin/chitosan powder dissolved in a 1% phosphate buffered saline or an appropriate solubalizing agent for approximately 15 minutes prior to the application of fibrinogen and thrombin.

To prepare the dressings, the temperature of the fibrinogen and thrombin is 4° C.±2° C. The moulds are removed from the −80° C. freezer and the dressings are placed on an aluminum tray on dry ice. A repeat pipettor filled with fibrinogen is prepared and filled with 5 ml of fibrinogen which is dispensed a total of four times, i.e., 20 ml. A second repeat pipettor is filled with 5 ml of thrombin and is dispensed a total of 3 times, i.e., 15 ml. Similarly repeat pipettors are used to dispense the kaolin and chitosan in solution onto the dressing. A total amount of 10 ml of kaolin and chitosan will be dispensed with the repeat pipettors in the concentrations mentioned above.

After application of the above mentioned compounds, the dressings is placed back in the −80° C. freezer for approximately 150 minutes. They are removal and lyophillised (freeze dried) and packed in foil for use.

In accordance with the disclosed subject matter, a fabric substrate is provided with a formulation of kaolin, chitosan, fibrinogen, and thrombin. For example, the formulation is impregnated or inter-dispersed or soaked into the fabric substrate to provide a medical device, such as a dressing or a bandage.

### Method of Action of Kaolin:

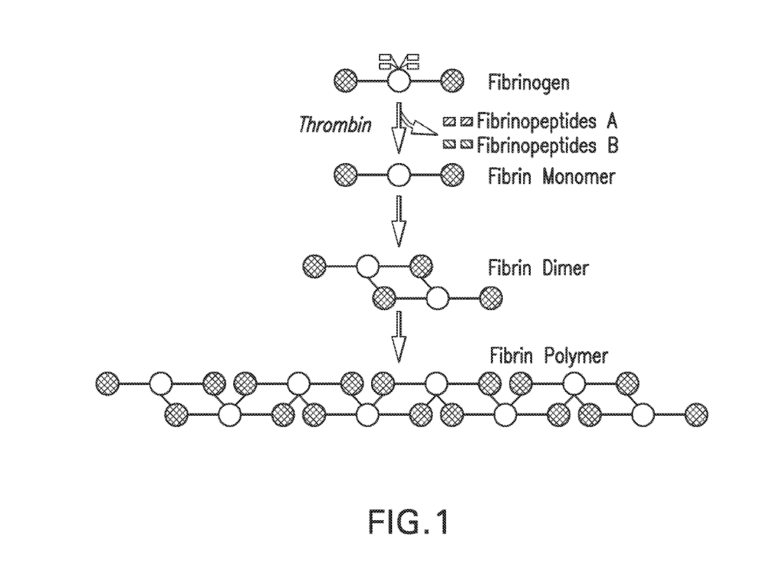

Contact between kaolin and Factor XII initiates the process. This reaction leads to the transformation of Factor XII, Factor XI and prekallikrein to their activated forms. Further, the activation of Factor XII to Factor XIIa is highly dependent on the presence of HMK and kaolin since both these compounds increase the susceptibility of Factor XII to plasma kallikrein. In addition, Factor XII can activate prekallikrein even without kaolin, but kaolin's presence markedly enhances the rate of activation. The activation of both Factor XI and XII will lead to the rest of the coagulation cascade as depicted in FIG. 1.

### Method of Action of Chitosan:

The method of action of chitosan is independent to that of the coagulation cascade. Additionally, chitosan has been shown to influence platelet aggregation.

### Method of Action of Thrombin and Fibrinogen:

As depicted in FIG. 1, thrombin converts fibrinogen to fibrin. Additionally fibrinogen also binds to proteins released by activated platelets. In one embodiment (“FORMULA 1”), the formulation is composed of a fibrinogen buffer (as shown below in TABLE 1), and a thrombin buffer (as shown below in TABLE 2):

FORMULA 1: Fibrinogen Buffer

| Component | Concentration |
|---|---|
| 100 mM TRIS Buffer | 16.76 g |
| 10 mM NaC1 | 2.94 g |
| 1.5% Sucrose | 15 g |
| 80 mg/g Fetal Bovine Serum (FBS) | 16 g |
| 15 mg/g Tween | 3.0 g |

Note that in the formulations mentioned in accordance with the present subject matter, it is possible to use bovine serum albumin (BSA) as an alternative for FBS. Tween is a brand name for polysorbates manufactured by ICI America's Inc, including polysorbate 20 and polysorbate 80. However, a polysorbate is manufactured by any manufacturer would be suitable.

The fibrinogen buffer is dissolved in 100 ml of 1% PBS to make the fibrinogen, 25 g of fibrinogen is mixed into the buffer at room temperature for approximately 40-60 min and the resultant solution is placed in a fridge at 4° C. This solution has a shelf-life of at least 2 weeks after it is made.

FORMULA 1: Thrombin Buffer

| Component | Concentration |
|---|---|
| 150 mM NaC1 | 8.72 g |
| 10 mM TRIS | 1.58 g |
| 40 mM CaC1 | 4.44 g |
| 100 mM LLysine | 18.76 g |
| 100 mg/g FBS | 18.76 g |

The thrombin buffer is dissolved in 100 ml of 1% PBS to make the thrombin buffer. Then 1000 KU of thrombin is added to this buffer and dissolved at room temperature for approximately 20 min and placed in a refrigerator or cooling system at 4 degrees centigrade. The solution is placed in a refrigerator or cooling system at 4° C. and has a shelf life of at least 2 weeks.

Also included in the composition of the exemplary “FORMULA 1” is 10 grams of kaolin which is dissolved in 200 ml of PBS by mixing at room temperature for approximately 15 minutes. For some embodiments, a range of 2-5 g of kaolin is used for each fabric substrate, e.g., bandage.

Also included in the composition of the exemplary “FORMULA 1” is 10 g of chitosan is dissolved in 200 ml of PBS. The solution is made acidic to Ph of less than 6.5 by addition of hydrochloric acid. The solution is then heated at 70° C. while being mixed. At approximately the 40 min time point the Ph is brought up to 7.4 by addition of sodium hydroxide. The solution is then allowed to cool, and a range of 4-10 g is applied to the bandage and placed in a −80° C. environment for approximately 15 minutes.

In accordance with another aspect of the disclosed subject matter, the method for preparing or manufacturing the bandage(s) includes placing the bandage in plastic pan, applying 2 g solution of Kaolin and 4 g solution of chitosan, and placing the bandage in −80° C. environment for approximately 15 minutes. Thereafter, fibrinogen is applied in a strength of 10 g and placed in a −80° C. environment for approximately 15 minutes. Then thrombin is applied in a strength of 100 KU per bandage and placed in a −80° C. environment for approximately 45 minutes. The bandage(s) are then placed in lyophilizer bottles. In the event the bandages are not yet frozen at the 45 minute time point after thrombin application then they can be dipped in liquid nitrogen before placing them into the lyophilizer. Thereafter, the bandage(s) are lyophilized for approximately 12 to 18 hours, such that they become malleable after the lyophilization process. Additionally, it may be advantageous in some applications to flatten out or otherwise reconfigure the lyophilized bandages to facilitate packaging.

In another exemplary embodiment (“FORMULA 2”), the formulation comprises 25 g of dissolved fibrinogen in 1% PBS at 37 degrees ° C. for approximately 60 minutes and agitated vigorously approximately every 15 minutes. This exemplary embodiment also includes the desired amount of thrombin (1000 KU) which is dissolved in either Glycine (at 2.5 g/100 ml of PBS); or glucose (at 5 g/100 mlPBS).

The method of preparation or manufacture of this exemplary “FORMULA 2” embodiment includes placing the bandage(s) in plastic pan or other container and applying 2 g solution of Kaolin, 4 g solution of chitosan, and placing the bandage(s) in −80° C. environment for approximately 15 minutes. Thereafter, fibrinogen is applied in a strength of 10 g and placed in a −80° C. environment for approximately 15 minutes. Then thrombin is applied in a strength of 100 KU per bandage and placed in a −80° C. environment for approximately 45 minutes. The bandage(s) are then placed in lyophilizer bottles. In the event the bandages are not yet frozen at the 45 minute time point after thrombin application then they can be dipped in liquid nitrogen before placing them into the lyophilizcr. Thereafter, the bandage(s) are lyophilized for approximately 12 to 18 hours, such that they become malleable after the lyophilization process. Additionally, it may be advantageous in some applications to flatten out or otherwise reconfigure the lyophilized bandages to facilitate packaging.

### Internal Application

In yet another exemplary embodiment, which is particularly suited for application internal to the patient's body, the formulation includes the formulation is composed of a fibrinogen buffer (as shown below in TABLE 3), and a thrombin buffer (as shown below in TABLE 4):

FORMULA 1 - Internal: Fibrinogen Buffer

| Component | Concentration |
|---|---|
| 100 mM TRIS Buffer | 16.76 g |
| 10 mM NaC1 | 2.94 g |
| 1.5% Sucrose | 15 g |
| 80 mg/g Human Serum | 16 g |
| 15 mg/g Tween | 3.0 g |

In certain applications, the fibrinogen buffer is dissolved in 100 ml of 1% PBS, and 25 g of human fibrinogen is added at room temperature for approximately 40-60 minutes. The resultant solution is then placed in a 4° C. environment and exhibits a shelf life of approximately 2 weeks.

FORMULA 1 - Internal: Thrombin Buffer

| Component | Concentration |
|---|---|
| 150 mM NaC1 | 8.72 g |
| 10 mM TRIS | 1.58 g |
| 40 mM CaCl | 4.44 g |
| 100 mM LLysine | 18.76 g |
| 100 mg/g Human Serum | 18.76 g |

In certain applications, the thrombin buffer is dissolved in 100 ml of 1% PBS, and 1000 KU of human thrombin is added and dissolved at room temperature for approximately 20 minutes. The resultant solution is then placed in a 4° C. environment and exhibits a shelf life of approximately 2 weeks.

Also included in the composition of the exemplary “FORMULA 1-Internal” is 10 grams of kaolin which is dissolved in 200 ml of PBS by mixing at room temperature for approximately 15 minutes. For some embodiments, a range of 2-5 g of kaolin is used for each fabric substrate, e.g., bandage.

Also included in the composition of the exemplary “FORMULA 1” is 20 g of chitosan is dissolved in 400 ml of PBS. The solution is made acidic to Ph of less than 6.5 by addition of hydrochloric acid. The solution is then heated at 70° C. while being mixed. At approximately the 40 min time point the Ph is brought up to 7.4 by addition of sodium hydroxide. The solution is then allowed to cool, and a range of 4-10 g is applied to the bandage and placed in a −80° C. freezer for approximately 15 min.

The method of preparation or manufacture of this exemplary “FORMULA 2” embodiment includes placing the bandage(s) in plastic pan or other container and applying 2-4 g solution of Kaolin, 4-10 g solution of chitosan, and placing the bandage(s) in −80° C. environment for approximately 15 minutes. Thereafter, fibrinogen is applied in a strength of 10-20 g and placed in a −80° C. environment for approximately 15 minutes. Then thrombin is applied in a strength of 100-200 KU per bandage and placed in a −80° C. environment for approximately 45 minutes. The bandage(s) are then placed in lyophilizer bottles. In the event the bandages are not yet frozen at the 45 minute time point after thrombin application then they can be dipped in liquid nitrogen before placing them into the lyophilizer. Thereafter, the bandage(s) are lyophilized for approximately 12 to 18 hours, such that they become malleable after the lyophilization process. Additionally, it may be advantageous in some applications to flatten out or otherwise reconfigure the lyophilized bandages to facilitate packaging.

In another embodiment (“FORMULA 2-Internal”), the formulation comprises 25-50 g of dissolved human fibrinogen in 1% PBS at 37 degrees ° C. for approximately 60 minutes and agitated vigorously approximately every 15 minutes. This exemplary embodiment also includes the desired amount of thrombin (1000-2000 KU) which is dissolved in either Glycine (at 2.5 g/100 ml of PBS); or glucose (at 5 g/100 mlPBS).

The method of preparation or manufacture of this exemplary “FORMULA 2-Internal” embodiment includes placing the bandage(s) in plastic pan or other container and applying 2-5 g solution of Kaolin, 4-10 g solution of chitosan, and placing the bandage(s) in a −80° C. environment for approximately 15 minutes. Thereafter, fibrinogen is applied in a strength of 10-20 g and placed in a −80° C. environment for approximately 15 minutes. Then thrombin is applied in a strength of 100-200 KU per bandage and placed in a −80° C. environment for approximately 45 minutes. The bandage(s) are then placed in lyophilizer bottles. In the event the bandages are not yet frozen at the 45 minute time point after thrombin application then they can be dipped in liquid nitrogen before placing them into the lyophilizer. Thereafter, the bandage(s) are lyophilized for approximately 12 to 18 hours, such that they become malleable after the lyophilization process. Additionally, it may be advantageous in some applications to flatten out or otherwise reconfigure the lyophilized bandages to facilitate packaging.

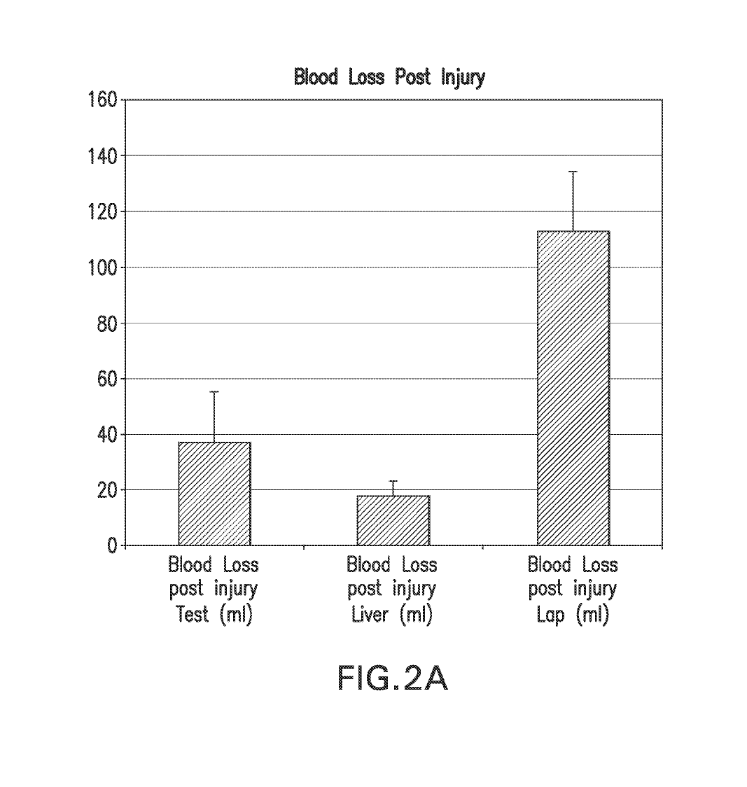

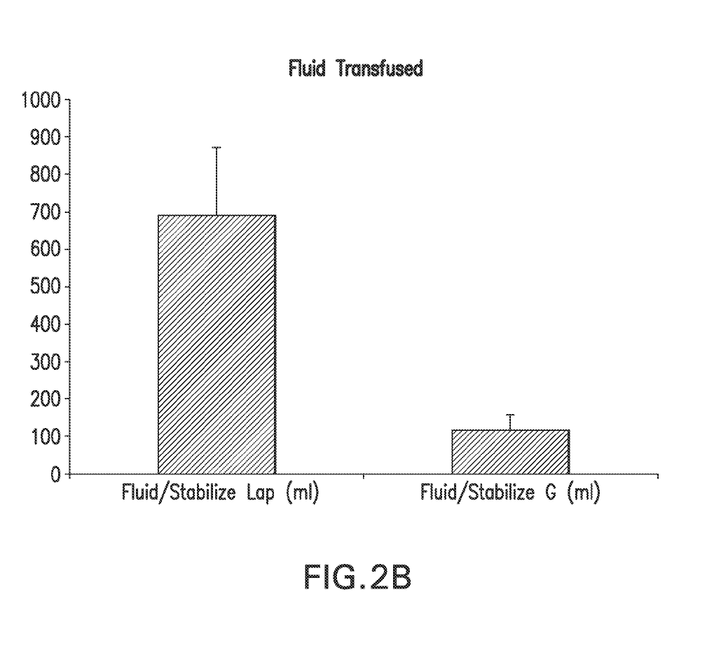

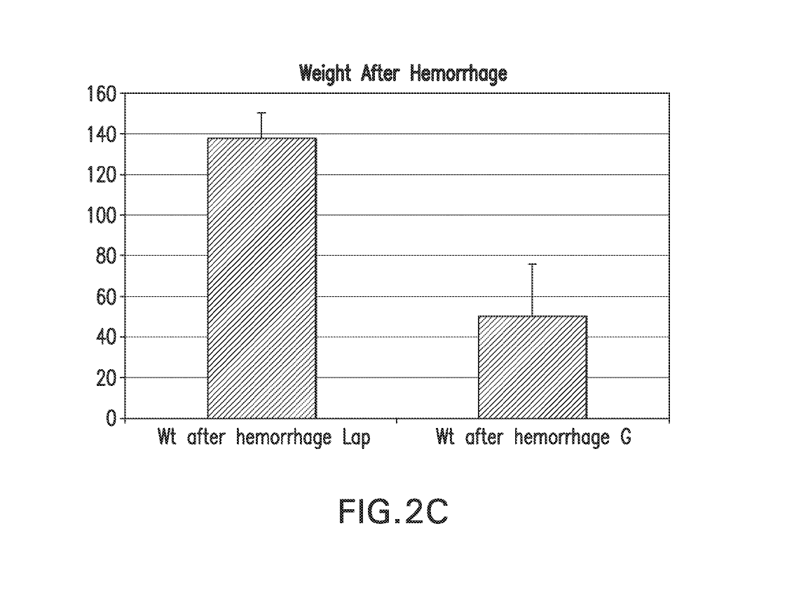

FIGS. 2A-2C illustrate the results of various tests conducted to measure the efficacy of the wound treatment system disclosed herein.

In addition to chitosan, kaolin, fibrinogen and thrombin, the wound treatment device and methods of the presently disclosed subject matter can include hemostatic compositions containing chamomile and nettle components. For purpose of illustration and not limitation, exemplary embodiments can include chamomile and nettle components in a ratio within the range of about 0.2-5.0:1.0 by weight of chamomile flowers to nettle leaves. Additional or alternative embodiments can employ a ratio of about 1.0-3.0 chamomile flowers to nettle leaves, as disclosed in U.S. Patent Application Publication No. 2008/0171074, the entirety of which is hereby incorporated by reference. These chamomile and nettle components may be provided by a variety of techniques (e.g. extraction) and other known and commercially available sources.

In accordance with another aspect of the disclosed subject matter, wound treatment methods and devices (e.g. bandage(s)) can include a biocompatible polymer or gel, which can be advantageous in impeding, absorbing, or otherwise collecting water. This action promotes coagulation, and can thus enhance the hemostatic effect of the presently disclosed subject matter. The amount and distribution of biocompatible polymer or gel utilized in a given bandage will ultimately be dictated by the intended application. A wide variety of biocompatible polymers are known in the art, for sake of brevity an exhaustive list is not provided herein though one of ordinary skill would recognize suitable polymers, gels and equivalents.

In accordance with yet another aspect of the disclosed subject matter, wound treatment methods and devices (e.g. bandage(s)) can include a pharmaceutical agent(s) to further aid and enhance the medicinal application of the bandage(s). For example, the bandage(s) can include one or more of an antimicrobial, an antibiotic, an antimicrobial, an antifungal, an antiviral, a local anesthetic, an analgesic, an antioxidant, an antiseptic agent, a vitamin, or combinations thereof. A wide variety of pharmaceutical agents are known in the art, for sake of brevity an exhaustive list is not provided herein though one of ordinary skill would recognize suitable pharmaceutical agents and equivalents.

In accordance with still another aspect of the disclosed subject matter, the bandage(s) of can be composed with a wide array of hemostatic capabilities including antiseptic, analgesic, anesthetic and/or anti-inflammatory properties. A wide variety of such agents are known in the art, for sake of brevity an exhaustive list is not provided herein though one of ordinary skill would recognize suitable antiseptic, analgesic, anesthetic and/or anti-inflammatory agents and equivalents.

As will be appreciated by those of ordinary skill in the art, the inventive hemostatic compositions may desirably be incorporated into any of a variety of formulations or devices for topical or transdermal administration. For example, although the exemplary embodiment disclosed relates to bandage(s), the novel features of the wound treatment system and methods disclosed herein are equally applicable to formulations as ointments, pastes, creams, lotions, gels, powders, solutions, sprays, or inhalants. Similarly, the wound treatment systems disclosed herein can be applied onto or incorporated within a surface, device, or material that is used to deliver the composition to a wound site. Likewise, the novel compositions disclosed herein can be applied evenly across substantially the entire surface of the material or configured for a localized application in areas of differing thickness. Similarly, the novel wound treatment system disclosed herein can be embodied into any desirable shape (e.g., square, rectangular, circular, or oval, or user-customizable). Moreover, the wound treatment devices can be sterilized and packaged, as is known in the art.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. It will be recognized by one of ordinary skill in the art that where a particular range or numerical value is provided, this is for purpose of illustration and not limitation, and alternative ranges and values are within the scope of the disclosed subject matter. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. A medical device, comprising:
a fabric substrate including a formulation, wherein the formulation includes chitosan, kaolin, fibrinogen, and thrombin.

2. The medical device of claim 1, wherein the fibrinogen is in an amount of about 25 to 50 grams, the thrombin is in an amount of about 1000 to 2000 kilo units, the kaolin is in an amount of about 2 grams to 10 grams, and the chitosan is in an amount of about 4 grams to 10 grams.

3. The medical device of claim 1, wherein the fibrinogen is a fibrinogen buffer solution, and the fibrinogen buffer solution further include tris(hydroxymethyl)aminomethane (TRIS), sodium chloride, sucrose, serum albumin, and polysorbate.

4. The medical device of claim 1, wherein the thrombin is a thrombin buffer solution, and the thrombin buffer solution includes sodium chloride, TRIS, calcium chloride, Lysine, and serum albumin.

5. The medical device of claim 1, wherein the kaolin is a kaolin solution, and the kaolin solution includes phosphate buffered saline (PBS).

6. The medical device of claim 1, wherein the chitosan is a chitosan solution, and the chitosan solution further includes PBS.

7. The medical device of claim 1, wherein the formulation has a pH of about 6 to 7.5.

8. The medical device of claim 1 wherein the fabric substrate is a dressing.

9. The medical device of claim 1, wherein the fabric substrate is a bandage.

10. (canceled)

11. The medical device of claim 1 wherein the fabric substrate comprises a composite textile.

12. The medical device of claim 1 wherein the fabric substrate has a size of approximately 1 to 3 inches in length and approximately 3 to 5 inches in width.

13. The medical device of claim 1 wherein the fabric substrate has a size of approximately 6 to 9 inches in length and approximately 8 to 11 inches in width.

14. A method for reducing bleeding from a wound site, comprising:
providing a medical device comprising a fabric substrate and a formulation comprising kaolin, fibrinogen, thrombin, and chitosan; and applying the medical device to the wound site.

15. A method for fabricating a hemostatic dressing, comprising;
providing a fabric substrate;
applying a kaolin solution to the fabric substrate;
applying a chitosan solution to the fabric substrate;
applying a fibrinogen solution to the fabric substrate; and
applying a thrombinogen solution to the fabric substrate.

16. (canceled)

17. (canceled)

18. (canceled)

19. (canceled)

20. A formulation useful to stop bleeding from a wound, the formulation comprising: chitosan, kaolin, fibrinogen, and thrombin.

21. The formulation of claim 20, wherein the fibrinogen is a fibrinogen buffer solution, and the fibrinogen buffer solution further includes TRIS, sodium chloride, sucrose, serum albumin and polysorbate.

22. The formulation of claim 20, wherein the thrombin is a thrombin buffer solution, and the thrombin buffer solution further includes sodium chloride, TRIS, calcium chloride, lysine and serum albumin.

23. The formulation of claim 20, wherein the thrombin is a thrombin solution, and the thrombin solution further includes glycine and glucose.

24. The formulation of claim 20, wherein the kaolin is a kaolin solution further including PBS.

25. The formulation of claim 20, wherein the chitosan is a chitosan solution further including PBS.
